(19) United States
(12) Patent Application Publication
(10) Pub. No.: US 2023/0357138 A1
(43) Pub. Date: Nov. 9, 2023

(54) SULFATIZED ESTERAMINES

(71) Applicant: BASF SE, Ludwigshafen am Rhein (DE)

(72) Inventors: Sophia EBERT, Ludwigshafen am Rhein (DE); Ouidad BENLAHMAR, Ludwigshafen am Rhein (DE); Dawid MARCZEWSKI, Ludwigshafen am Rhein (DE); Dieter BOECKH, Ludwigshafen am Rhein (DE); Christine MENDERA, Dannstadt-Schauernheim (DE)

(21) Appl. No.: 18/013,274

(22) PCT Filed: Jun. 24, 2021
(86) PCT No.: PCT/EP2021/067396

§ 371 Date: Dec. 28, 2022

(30) Foreign Application Priority Data
Jul. 3, 2020 (EP) 20183941.2

(51) Int. Cl. C07C 309/10

(52) U.S. Cl. CPC C07C 309/10

(57) ABSTRACT

The present invention relates to sulfatized esteramines obtainable by a process comprising step a), wherein at least one alcohol containing at least two hydroxy groups (compound (A)) is reacted with at least one lactam (compound (B)) and with sulfuric acid (compound (C)). The present invention also relates to a process for preparing such sulfatized esteramines.

The present invention relates to sulfatized esteramines obtainable by a process comprising step a), wherein at least one alcohol containing at least two hydroxy groups (compound (A)) is reacted with at least one lactam (compound (B)) and with sulfuric acid (compound (C)). The present invention also relates to a process for preparing such sulfatized esteramines.

WO 2019/007750 relates to alkoxylated esteramines and salts thereof according to a specific formula (I). In case the respective compound is a salt, the respective salt may be obtained by at least partial protonation of the amine groups contained within the compounds according to formula (I) by an acid selected from compounds such as methane sulfonic acid, hydrochloric acid, sulfuric acid, phosphoric acid or lactic acid. The alkoxylated esteramines of WO 2019/007750 may be obtained by reacting at least one alcohol according to a specific formula (III) with at least one C₂ to C₁₆ alkylene oxide followed by at least partial esterification with at least one aminoacid such as alanine, lysine or an acid according a specific formula (IV). Further alkoxylated esteramines and salts thereof are disclosed within WO 2019/007754.

WO 2019/110371 relates to a process for the preparation of organic sulfonic salts of aminoacid esters as well as to the organic sulfonic acid salts of aminoacid esters as such. The respective organic sulfonic acid salts of aminoacid esters are obtained by a process comprising the reaction of at least one lactam having at least three carbon atoms in the lactam ring with at least one organic sulfonic acid in aqueous solution (step i)) and the esterification of the reaction product of step i) with at least one alcohol with at least 8 carbon atoms comprising at least one hydroxyl group.

European application 19150654.2 relates to a process for the preparation of organosulfate salts of aminoacid esters within the respective process. At least one lactam with at least 3 carbon atoms in the lactam ring is reacted with sulfuric acid within the first step and an esterification of the reaction product of the first step with at least 200 mol-% of at least one alcohol containing only one hydroxy group is carried out in a 35 second step.

CN 109 880 079 A and CN 108 774 318 A relate to a process for the production of antibacterial and antistatic or antistatic Nylon-6, respectively, wherein caprolactam and deionized water (and optionally nano-silver) are added into a autoclave in a first step and the resulting solution is reacted with ethylenglycol or polyethylenglycol and concentrated sulfuric acid in a second step. In a third step, further caprolactam and a molecular weight regulator are added to the polymerization reactions. Furthermore, Khitrin et al.: Reactions of epsilon-caprolactam with alcohols, Database Caplus accession no. 1997:465734 relates to the reactions of ε-caprolactam with alcohols.

The object is achieved by a sulfatized esteramine obtainable by a process comprising step a):
a) reacting at least one alcohol containing at least two hydroxy groups (compound (A)) with at least one lactam (compound (B)) and with sulfuric acid (compound (C)).

The sulfatized esteramines according to the present invention may be used in specific compositions, such as detergent, cleaning and/or fabric and home care compositions/formulations.

An advantage can be seen in the fact that the sulfatized esteramines according to the present invention show improved clay dispersing properties and/or an improved whiteness compared to esteramines based on, for example, alkoxylated and non-alkoxylated di- and polyols without sulfate groups. This means, expressed in other words, that the respective esteramines according to the prior art do not mandatorily contain any OSO₃ fragments.

The invention is specified in more detail as follows:

The invention relates to a sulfatized esteramine obtainable by a process comprising step a):
a) reacting at least one alcohol containing at least two hydroxy groups (compound (A)) with at least one lactam (compound (B)) and with sulfuric acid (compound (C)).

Generally, as used herein, the term “obtainable by” means that corresponding products do not necessarily have to be produced (i.e. obtained) by the corresponding method or process described in the respective specific context, but also products are comprised which exhibit all features of a product produced (obtained) by said corresponding method or process, wherein said products were actually not produced (obtained) by such method or process. However, the term “obtainable by” also comprises the more limiting term “obtained by”, i.e. products which were actually produced (obtained) by a method or process described in the respective specific context.

When used herein any definition requiring a compound or a substituent of a compound to consist of “at least a number of carbon atoms”, number of carbon atoms refers to the total number of carbon atoms in said compound or substituent of a compound. For example for a substituent disclosed as “alkyl ether with at least 8 carbon atoms comprising alkylene oxide groups”, the total number of at least 8 carbon atoms needs to be the sum of the number of carbon atoms of the alkyl moiety and the number of carbon atoms of the alkylene oxide moieties.

The term “containing at least two hydroxy groups” means that two or more —OH groups are present. The term “hydroxy group” is equal to the term “hydroxyl group” or “—OH group”. Alcohols/compounds having only one hydroxy group, such as methanol or ethanol, do, by consequence, not fall under the definition of an alcohol containing at least two hydroxy groups according to compound (A) of the present invention. Any functionalized group derived from a hydroxy group such as an ether group is not considered to be an —OH group.

Alcohols containing at least two hydroxy groups according to compound (A) are known to a person skilled in the art. As mentioned above, the respective alcohol may contain two, three, four, five or even more hydroxy groups within the respective molecule/compound. The respective alcohol may contain linear, branched and/or cyclic alkyl fragments. Beyond that, the respective alcohol may also contain aromatic fragments as well as combinations of alkyl and aromatic fragments (“aralkyl fragments”). Furthermore, the respective alcohol may also contain alkyl ether fragments. Examples of alcohols according to compound (A) are glycerol, pentaerythrit, sorbitol, 1,1,1-trimethylolpropane (TMP) or alkoxylated alcohols, such as polyethylene glycol. Alcohols according to compound (A) of the present invention are usually commercially available, for example, under the tradename “Pluronics” (for example as polyethyleneglycol block (co)polymers) from BASF SE.

In one embodiment of the present invention, at least one linear or branched C₂- to C₃₆-alcohol containing at least two hydroxy groups is used.

In another embodiment, alkylether alcohols are used. Alkylether alcohols are for example alkyl alcohols alkoxylated with ethylene oxide, and/or propylene oxide, and/or butylene oxide. In one embodiment of the present invention, at least one linear or branched C₂- to C₃₆-alcohol containing at least two hydroxy groups alkoxylated with ethylene oxide, and/or propylene oxide, and/or butylene oxide is used. In another embodiment at least one C₈- to C₂₂-alcohol containing at least two hydroxy groups alkoxylated with ethylene oxide, and/or propylene oxide, and/or butylene oxide is used.

Alkoxylation of the alcohol is either carried out with only one alkylene oxide or with more than one alkylene oxide. If more than one alkylene oxide is used, the resulting alkylether alcohols comprises either randomly distributed alkylene oxide units or a block of one alkylene oxide followed by a block of another alkylene oxide. In one embodiment of the present invention, alkyl alcohols alkoxylated with only a single alkylene oxide are used. In a further embodiment, alkyl alcohols alkoxylated with a first alkylene oxide followed by alkoxylation with a second alkylene oxide, thereby forming a block structure of different alkylene oxide blocks, are used.

The at least one alcohol containing at least two hydroxy groups according to compound (A) is preferably at least one alcohol containing at least two hydroxy groups selected from diols, polyols, alkoxylated diols and alkoxylated polyols,
more preferably, selected from sorbitol, 1,6-hexanediol, glycerol, 1,1,1-trimethylolpropan (TMP), pentaerythrit, polyethyleneglycol, ethylene glycol, alkoxylated ethylene glycol, propylene glycol, alkoxylated propylene glycol, polypropylene glycol, alkoxylated sorbitol, alkoxylated 1,6-hexanediol, alkoxylated glycerol, alkoxylated TMP and alkoxylated pentaerythrit,
most preferably, selected from 1,6-hexanediol, alkoxylated sorbitol, alkoxylated glycerol, polyethylene glycol, alkoxylated TMP and alkoxylated pentaerythrit.

Within the context of the present invention, it is also preferred that in case compound (A) comprises an alkoxylated alcohol containing at least two hydroxy groups, the alkoxylated fragment of the respective alcohol is based on at least one C₂-C₂₂ alkylene oxide, more preferably on ethylene oxide and/or propylene oxide, most preferably the respective alcohol comprises at least one block based on ethylene oxide and/or propylene oxide.

Within the context of the present invention, it is also preferred that in case an alkoxylated alcohol containing at least two hydroxy groups is employed as compound (A), the respective alkoxylation in order to obtain the respective alkoxylated alcohol is carried out prior to step a) as a separate step b). Expressed in other words, this means that first an alkoxylated alcohol according to compound (A) is prepared and, for example, directly afterwards, the respective alkoxylated alcohol is employed within step a) of the process according to the present invention in order to obtain the sulfatized ester amines according to the present invention.

It is therefore preferred that the sulfatized ester amines according to the present invention are obtainable by a process comprising steps a) and b), wherein step a) is defined as above and the process also comprises step b), which is carried out prior to step a):
b) at least one alcohol containing at least two hydroxy groups and having a molecular weight M_{w} of less than 500 g/mol is reacted with at least one alkylene oxide in order to obtain an alkoxylated alcohol as compound (A).

Within step b), it is even more preferred that the sulfatized esteramine according to the present invention is obtained, wherein
i) ethylene oxide and/or propylene oxide is employed, and/or
ii) at least one alcohol containing at least two hydroxy groups and having a molecular weight M_{w} of less than 500 g/mol is reacted with at least 1 mol of propylene oxide and/or with at least 1 mol of ethylene oxide, and/or
iii) at least one alcohol containing at least two hydroxy groups and having a molecular weight M_{w} of less than 500 g/mol is reacted batchwise with ethylene oxide and/or propylene oxide in order to obtain at least one block based on ethylene oxide and/or propylene oxide on the respective alkoxylated alcohol, and/or
iv) at least one alcohol containing at least two hydroxy groups and having a molecular weight M_{w} of less than 500 g/mol is reacted in at least one batch with 1 to 120 mol of propylene oxide followed by at least one batch of 1 to 150 mol ethylene oxide.

The at least one lactam according to compound (B) is known to a person skilled in the art. In principle, any lactam which is stable and known to a person skilled in the art can be employed as compound (B) within the context of the present invention.

Lactams are cyclic amides, starting with α-lactam (three ring atoms) followed by β-lactam (four ring atoms), γ-lactam (five ring atoms) and so on. When hydrolyzed, lactams form the corresponding α-, β-, γ-amino acid. All lactams with at least three carbon atoms in the lactam ring can be used in the process for the synthesis of sulfatized esteramines according to the present invention. In one embodiment of the present invention, lactams with of from four to twelve carbon atoms in the lactam ring are used. In another embodiment of the present invention, lactams with of from five to seven carbon atoms in the lactam ring are used. In a further embodiment, a lactam with six carbon atoms in the lactam ring, ε-lactam, is used.

Reaction of the lactam ring may take place by reacting the at least one lactam with sulfuric acid. Reaction of the lactam ring with the sulfuric acid is preferably carried out in an aqueous solution. In one embodiment of the present application the reaction of the lactam ring takes place by reacting the at least one lactam with sulfuric acid in an aqueous solution containing only water.

The term “free of water” means that the composition contains no more than 5 wt.-% of water based on the total amount of solvent, in another embodiment no more than 1 wt.-% of water based on the total amount of solvent, in a further embodiment the solvent contains no water at all.

The term “aqueous solution” means that the solvent contains more than 50 wt.-% of water based on the total amount of solvent. In a further embodiment the term means that the solvent contains more than 80 wt.-% of water based on the total amount of solvent. In another embodiment the term means that the solvent contains more than 95 wt.-% of water based on the total amount of solvent. In a further embodiment the term means that the solvent contains more than 99 wt.-% of water based on the total amount of solvent. In an even further embodiment the term means that the solvent contains only water.

Within the present invention, it is preferred that compound (B) is at least one ε-lactam, most preferably caprolactam.

Sulfuric acid as such, which is employed as compound (C) within the present invention, is known to a person skilled in the art.

In one embodiment of the present invention, the lactam is selected from the group consisting of a lactam with five carbon atoms in the lactam ring, and a lactam with six carbon atoms in the lactam ring, and the reaction with sulfuric acid is carried out in an aqueous solution. In another embodiment of the present invention, the lactam has five carbon atoms in the lactam ring and the reaction with sulfuric acid is carried out in an aqueous solution.

In one embodiment the lactam is either dissolved in water or is dispersed in an aqueous phase. Typical concentration of lactam in water is in the range of from 50% by weight to 99% by weight based on the total weight of lactam and water. In one embodiment of the present invention the concentration of lactam in water is in the range of from 55 to 90% by weight based on the total weight of the lactam and water. In a further embodiment the concentration of lactam in water is in the range of from 65 to 80% by weight based on the total weight of the lactam and water.

In one embodiment, sulfuric acid is used as concentrated sulfuric acid. In another embodiment, sulfuric acid is used as 96 to 98 wt.-% sulfuric acid solution in water. In a further embodiment sulfuric acid is used as 80 wt.-% sulfuric acid solution in water.

In one embodiment of the present invention the total amount of sulfuric acid is added at the beginning of the reaction to the at least one lactam. In another embodiment the sulfuric acid is added dropwise for a duration of from 0.1 to 10 h to the at least one lactam.

The process as such comprising step a) in order to obtain the sulfatized esteramines according to the present invention can be carried out by any method known to a person skilled in the art. Specific ways/embodiments for carrying out step a) according to the present invention, are described in further detail below within the experimental section.

Step a) according to the present invention may be carried out by mixing the respective compounds (A) to (C) in any order and/or sequence. For example, it is possible to mix all three components together before starting the reaction as such. However, it is also possible to mix only parts of these components in advance and the remaining parts of the respective components or even the complete part of a single component afterwards. For example, step a) can also be carried out batchwise and/or continuously.

Within the context of the present invention, it is preferred that in step a)
i) at least a fraction of compound (A) is first mixed with at least a fraction of compound (B) followed by continuously adding at least a fraction of compound (C) over a specific period of time, preferably the entire amount of compound (A) is first mixed with the entire amount of compound (B) followed by continuously adding the entire amount of compound (C), and/or
ii) compound (C) is added for a specific period of time and the specific period of time for continuously adding compound (C) is preferably in the range of less than one hour, more preferably less than 30 minutes, most preferably between 5 and 15 minutes, and or
iii) the reaction is carried out after all compounds (A) to (C) are admixed with each other at a temperature of 80 to 200° C. and/or water is removed from the reaction mixture.

The specific ratio of the individual compounds (A) to (C) can, in principle, be freely chosen. However, it is preferred that at least one of the following conditions, preferably all of the following conditions, is fulfilled when carrying out step a) according to the present invention.

It is preferred within the context of the present invention that in step a) the molar ratio of compound (C) to compound (B) is at least 100 mol-%, preferably in the range of 100 mol-% to 125 mol-%.

In another embodiment within the context of the present invention the molar ration of compound (C) to compound (B) in step a) is at least 90 mol-%, preferably in the range of 90 mol-% to 125 mol-%.

It is preferred within the context of the present invention that in step a) the molar ratio of compound (B) to the hydroxy groups of compound (A) is in the range of 10 mol-% to 50 mol-%.

It is preferred within the context of the present invention that in step a) the molar ratio of compound (C) to the hydroxy groups of compound (A) is in the range of 10 mol-% to 62.5 mol-%.

Within the context of the present invention, it is even more preferred that in step a) at least 10% of all hydroxy groups of compound (A) are reacted with compound (B) in order to form ester groups within the respective sulfatized esteramine and/or at least 10% of all hydroxy groups of compound (A) are sulfatized in order to form OSO₃ fragments within the respective sulfatized esteramine.

It is even more preferred that in step a)
20 to 50% of all hydroxy groups of compound (A) are reacted with compound (B) in order to form ester groups within the respective sulfatized esteramine,
20 to 50% of all hydroxy groups of compound (A) are sulfatized in order to form OSO₃ fragments within the respective sulfatized esteramine, and
0 to 30% of all hydroxy groups of compound (A) remain in unreacted form within the respective sulfatized esteramine.

As already mentioned above, it is also possible that besides compounds (A) to (C), further compounds such as solvent and/or water are present when carrying out step a). In addition, it is also possible that prior to and/or after step a), further steps may be carried out in order to obtain the sulfatized esteramines according to the present invention.

In one embodiment of the present invention, step a) is carried out in the presence of at least one solvent and/or in the presence of water. It is preferred that step a) is carried out in the presence of water, preferably by employing an aqueous solution of compound (B).

It is also preferred that in case a solvent and/or water is employed and/or in order to remove an excess of unreacted educts that an additional step (C) is carried out after step a) is finished. However, it is also possible that step c) is already started in parallel to performing step a) or at the end of performing step a).

In one embodiment of the present invention, it is preferred that an optional step c) is carried out by removing water and/or by removing excess alcohol according to compound (A), preferably step c) is carried out after step a) is finished.

By consequence, within step c) of the present invention, water and/or excess alcohol can be removed. Removal of water and alcohol can be carried out by all techniques known in the art, for example by application of a vacuum. In one embodiment of the present invention step c), the optional removal of water and/or excess of alcohol, is carried out applying a vacuum in the range of from 0.1 mbar to 800 mbar. In another embodiment vacuum in the range of from 1 mbar to 500 mbar is applied. In a further embodiment vacuum in the range of from 10 mbar to 100 mbar is applied.

Within the context of the present invention, it is preferred that step a) is carried out by
i) the reaction is carried out after all compounds (A) to (C) are admixed with each other at a temperature of 80 to 200° C. for a period of time of 1 to 30 hours, and/or
ii) the reaction is carried out in a closed vessel under pressure from 1.0 up to 10 bar, preferably 1.0 to 5 bar, most preferably 1.0 to 4 bar.

In another embodiment of the present invention, step a) is carried out by a process comprising steps i) to iii):
(i) reacting at least one lactam with at least 3 carbon atoms in the lactam ring with sulfuric acid;
(ii) esterification of the reaction product of step (i) with 10-50 mol-% of the hydroxy groups of an alcohol containing at least two hydroxy groups:
(iii) optionally removal of water and/or removal of excess alcohol of step (ii).

Within this embodiment of the present invention, step a) is carried out in accordance with the specific sequence of steps as disclosed within EP application 19150654.2 (in respect of steps i) to iii)). Moreover, this embodiment of the present invention differs from the respective disclosure of EP application 19150654.2 in the definition of the alcohol, which is within the context of the present invention an alcohol according to component (A) as defined above, whereas in EP application 19150654.2 an alcohol mandatorily containing only one hydroxyl group is employed in the respective process.

In another embodiment, the present invention relates to sulfatized esteramines of Formula (I) and salts thereof,

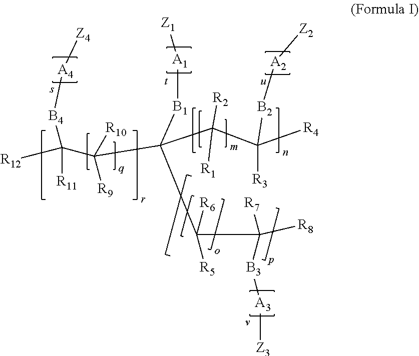

wherein independently from each other
n being an integer from 1 to 12,
m being an integer for each repetition unit n independently selected from 0 to 12;
p being an integer from 0 to 12,
o being an integer for each repetition unit p independently selected from 0 to 12;
r being an integer from 0 to 12,
q being an integer for each repetition unit r independently selected from 0 to 12;
s, t, u and v being an integer from 0 to 100;
A₁, A₂, A₃, and A₄ are independently from each other and independently for each repetition unit s, t, u, or v, selected from the list consisting of alkyleneoxy group, such A-units stem from the reaction of one alcohol with at least two hydroxy groups with C2-C22 alkylene oxides, e.g. in case of ethoxylated alcohols with at least two hydroxy groups A is “—O—CH2—CH2—”
wherein for s, t, u, and/or v equal to 1 the oxygen atom of the A₁, A₂, A₃, and A₄ group is bound to the B group and the following A₁, A₂, A₃, and A₄ groups are always bound via the oxygen atom to the previous A₁, A₂, A₃, and A₄ group.
B₁, B₂, B₃, and B₄ are independently from each other selected from the group consisting of a bond, linear C₁ to C₁₂ alkanediyl groups, and branched C₁ to C₁₂ alkanediyl groups;
such B-units are given by the molecular structure of one alcohol with at least two hydroxy groups, e.g. in case of example 2 (1,6-hexane diol, esterified with 1 mol caprolactam and esterified with 1 mole sulfuric acid) B1 and B2 are “—CH2—”, with p and r=0, n=1, m=2, t and u=0, R₁, R₂, R₃ R₄, R₈, R₉, and R₁₂=H, Z1 and Z2=OSO3H, OH or Formula (II) with w=3, and R₁₃, R₁₄ R₁₅, R₁₆, R₁₇, and R₁₈=H, in case of example 6 (ethoxylated glycerol, esterified with 1 mol caprolactam and esterified with 1 mole sulfuric acid) B₁, B₂ and B₄ are “-”, with p=0, r=1, n=1, m=0, q=0, t=4, u=4, s=4; R₃ R₄, R₈, R₁₁, and R₁₂=H; Z₁, Z₂ and Z₄=OSO₃H, OH or Formula (II) with w=3, and R₁₃, R₁₄ R₁₅, R₁₆, R₁₇, and R₁₈=H,
R₁, R₂, R₃ R₄, R₅, R₆, R₇, R₈, R₉, R₁₀, R₁₁ and R₁₂ being independently for each 20 repetition unit selected from the group consisting of H, linear alkyl, branched alkyl, and cycloalkyl; such R-units are given by the molecular structure of one alcohol with at least two hydroxy groups,
Z₁, and/or Z₂, and/or Z₃, and/or Z₄, independently for each repetition unit n, p, and r, are selected from the group consisting of OH, and OSO3—, and —OSO3H and a compound according to Formula (II), wherein said compound according to Formula (II) connects to the compound according to Formula (I) via the bond labeled with *, such Z-units stem from the reaction of one alcohol with at least two hydroxy groups with at least one lactam and with sulfuric acid, e.g. in case of reaction with C4 lactam and sulfuric acid, Z₁, Z₂, Z₃, Z₄, are “—OC(O)—CH₂—CH₂—CH₂—NH₂ or SO₃H or OH
with the provisio that at least 10 mol % to 50 mol % of the substituents Z₁, and/or Z₂, and/or Z₃, and/or Z₄, are a compound according to Formula (II), and at least 10 mol % to 50 mol % of the substituents Z₁, and/or Z₂, and/or Z₃, and/or Z₄, are a group consisting OSO3—,or —OSO3H, and 0 mol % to 80 mol % of the substituents Z₁, and/or Z₂, and/or Z₃, and/or Z₄, are OH,

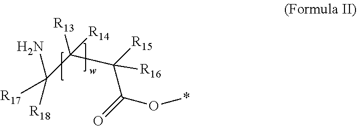

with independently from each other
w being an integer from 0 to 12;
R₁₃, R₁₄ R₁₅, R₁₆, R₁₇, and R₁₈ independently being selected from the group consisting of H, linear alkyl, branched alkyl, and cycloalkyl; such R-units stem from the lactam, e.g. in case of reaction with C4 lactam R₁₃, R₁₄ R₁₅, R₁₆, R₁₇, and R₁₈ are=H, w=1, and then Formula (II) is “—OC(O)—CH₂—CH₂—CH₂—NH₂”

The sulfatized esteramines according to the above defined formula (I) or the respective salts thereof are obtained by the process as described above. The definition of the the sulfatized esteramines according to formula (I) is a result of an optimized way for carrying out the respective process, wherein all functional groups (of the respective monomers or any intermediate) have undergone a complete reaction. A complete reaction (the conversion degree of 100%) is an idealized assumption. In reality, the degree of conversion is usually below 100%. Unreacted hydroxy groups may be present. This fact is known to a person skilled in the art due to the complexity of the reaction as well as the structure according to formula (I). Irrespective of that, the reaction for obtaining said structure is disclosed in the description above. By following the general reaction conditions as well as knowing specific reaction conditions, the real structure for each individual case/reaction condition is obvious for a person skilled in the art

Another subject of the present invention is also the process as such for producing these sulfatized esteramines as described above, wherein the process comprises step a):
a) reacting at least one alcohol containing at least two hydroxy groups (compound (A)) with at least one lactam (compound (B)) and with sulfuric acid (compound (C)).

It is obvious for a person skilled in the art that the process as such can be carried out analogously as described above for the first subject matter of the present invention, the sulfatized esteramines as such obtainable by a process comprising step a) including all variations and/or embodiments and/or preferred definitions.

Another subject matter of the present invention is the use of the above-mentioned sulfatized esteramines in cosmetic formulations, as crude oil emulsion breaker, in pigment dispersions for ink jet inks, formulations for electro plating, in cementitious compositions.

The inventive sulfatized esteramines can be added to cosmetic formulations, as crude oil emulsion breaker, in pigment dispersions for ink jet inks, formulations for electro plating, in cementitious compositions. However, the inventive compounds can also be added to (used in) washing or cleaning compositions.

The inventive sulfatized esteramines are present in said formulations at a concentration of 0.1 to 5 weight %, preferably at a concentration of 0.5 to 2 weight %.

The inventive sulfatized esteramines can also be added to a cleaning composition comprising from about 1% to about 70% by weight of a surfactant system. The inventive sulfatized esteramines may be present in a cleaning composition at a concentration of from about 0.1% to about 5% by weight of the composition, or at a concentration of from about 0.5% to about 2% by weight of the composition.

### Cleaning Composition

As used herein the phrase “cleaning composition” includes compositions and formulations designed for cleaning soiled material. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, dish washing compositions, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation. The cleaning compositions may have a form selected from liquid, powder, single-phase or multi-phase unit dose, pouch, tablet, gel, paste, bar, or flake.

The cleaning compositions comprise a surfactant system in an amount sufficient to provide desired cleaning properties. In some embodiments, the cleaning composition comprises, by weight of the composition, from about 1% to about 70% of a surfactant system. In other embodiments, the liquid cleaning composition comprises, by weight of the composition, from about 2% to about 60% of the surfactant system. In further embodiments, the cleaning composition comprises, by weight of the composition, from about 5% to about 30% of the surfactant system. The surfactant system may comprise a detersive surfactant selected from anionic surfactants, nonionic surfactants, cationic surfactants, zwitterionic surfactants, amphoteric surfactants, ampholytic surfactants, and mixtures thereof. Those of ordinary skill in the art will understand that a detersive surfactant encompasses any surfactant or mixture of surfactants that provide cleaning, stain removing, or laundering benefit to soiled material.

The cleaning compositions may also contain adjunct cleaning additives. Suitable adjunct cleaning additives include builders, structurants or thickeners, clay soil removal/anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, hueing agents, dye transfer inhibiting agents, chelating agents, suds supressors, softeners, and perfumes.

The following examples shall further illustrate the present invention without restricting the scope of the present invention.

### Example 1: Sorbitol, Propoxylated with 96 Mole Propylene Oxide and Ethoxylated with 144 Mole Ethylene Oxide, Esterified with 2 Mole Caprolactam and Sulfatized with 2 Mole Sulfuric Acid

### 1a Sorbitol, Propoxylated with 18 Mole Propylene Oxide

In a 2 l autoclave 248.9 g sorbitol and 6.6 g potassium hydroxide (50% in water) are placed and the mixture is heated to 120° C. Vacuum is applied and the mixture is stirred for 2 hours under vacuum (<10 mbar). The vessel is filled with nitrogen and heated to 140° C. 1400.0 g propylene oxide is added in portions within 40 h. To complete the reaction, the mixture is allowed to post-react for additional 10 h at 140° C. The reaction mixture is stripped with nitrogen and volatile compounds are removed in vacuo at 80° C. After filtration 1635.0 g of a brown oil is obtained (hydroxy value: 262 mgKOH/g).

### 1b Sorbitol, Propoxylated with 96 Mole Propylene Oxide

In a 2 l autoclave 180.0 g sorbitol, propoxylated with 18 mole propylene oxide and 3.4 g potassium hydroxide (50% in water) are placed and the mixture is heated to 110° C. Vacuum is applied and the mixture is stirred for 2 hours under vacuum (<10 mbar). The vessel is filled with nitrogen and heated to 140° C. 665.9 g propylene oxide is added in portions within 6 h. To complete the reaction, the mixture is allowed to post-react for additional 6 h at 140° C. The reaction mixture is stripped with nitrogen and volatile compounds are removed in vacuo at 80° C. After filtration 836.0 g of a light brown oil is obtained (hydroxy value: 58.5 mgKOH/g).

### 1c Sorbitol, Propoxylated with 96 Mole Propylene Oxide and Ethoxylated with 144 Mole Ethylene Oxide

In a 2 l autoclave 432.7 g sorbitol, propoxylated with 96 mole propylene oxide are placed and the mixture is heated to 60° C. The vessel is purged three times with nitrogen and heated to 140° C. 475.7 g ethylene oxide is added in portions within 4 hours. To complete the reaction, the mixture is allowed to post-react for additional 6 hours at 140° C. The reaction mixture is stripped with nitrogen and volatile compounds are removed in vacuo at 80° C. After filtration 883.0 g of a viscous brown waxy solid is obtained (hydroxy value: 27.8 mgKOH/g).

### 1d Sorbitol, Propoxylated with 96 Mole Propylene Oxide and Ethoxylated with 144 Mole Ethylene Oxide, Esterified with 2 Mole Caprolactam and Sulfatized with 2 Mole Sulfuric Acid

In a 4-neck vessel with thermometer, nitrogen inlet, dropping funnel, reflux condenser and stirrer 157.3 g sorbitol, propoxylated with 96 mole propylene oxide and ethoxylated with 144 mole ethylene oxide (1 c), 3.7 g caprolactam (80% in water), and 1.8 g water are placed. To the mixture 2.7 g sulfuric acid (96%) is added within 10 minutes under a constant stream of nitrogen. Temperature rises up to 55° C. during sulfuric acid addition. The reaction mixture is heated to 135° C. bath temperature and is stirred for 10 hours at 135° C. under reflux. The reflux condenser is removed and under a constant stream of nitrogen, water is distilled off for 3 hours. 160.0 g of a brown solid is obtained. ¹H-NMR in MeOD indicates 25% conversion of hydroxyl groups into 6-aminohexane acid ester and 33% conversion of hydroxyl groups into sulfuric acid ester.

### Example 2: 1,6-Hexane Diol, Esterified with 1 Mole Caprolactam and Esterified with 1 Mole Sulfuric Acid

In a 4-neck vessel with thermometer, nitrogen inlet, dropping funnel, reflux condenser and stirrer 23.6 g 1,6-hexane diol, 28.3 g caprolactam (80% in water), and 12.3 g water are placed. To the mixture 20.8 g sulfuric acid (96%) is added within 10 minutes under a constant stream of nitrogen. Temperature rises up to 50° C. during sulfuric acid addition. The reaction mixture is heated to 135° C. bath temperature and is stirred for 3 hours at 135° C. under reflux. The reflux condenser is removed and under a constant stream of nitrogen, water is distilled off for 2 hours. 60.0 g of a brown solid is obtained. ¹H-NMR in MeOD indicates 45% conversion of hydroxyl groups into 6-aminohexane acid ester and 40% conversion of hydroxyl groups into sulfuric acid ester.

### 5 Example 3: 2-Butyl-2-ethyl-1,3-propane diol, Esterified with 1 Mole Caprolactam and Esterified with 1 Mole Sulfuric Acid

In a 4-neck vessel with thermometer, nitrogen inlet, dropping funnel, reflux condenser and stirrer 81.5 g molten 2-Butyl-2-ethyl-1,3-propane diol, 72.0 g caprolactam (80% in water), are placed at 30° C. To the mixture 52.4 g sulfuric acid (96%) is added within 10 minutes. Temperature rises up to 65° C. during sulfuric acid addition. The reaction mixture is heated to 135° C. bath temperature and is stirred for 3 hours at 135° C. under reflux. The reflux condenser is removed and under a constant stream of nitrogen, water is distilled off for 2 hours. 180.0 g of a light yellow highly viscous oil is obtained. ¹H-NMR in MeOD indicates 47% conversion of hydroxyl groups into 6-aminohexane acid ester and 35% conversion of hydroxyl groups into sulfuric acid ester.

### Example 4: Polyethylene Glycol, Molecular Weight 4000 g/mol, Esterified with 1 Mole Caprolactam and Esterified with 1 Mole Sulfuric Acid

In a 250 ml glass pressure vessel with magnetic stir bar 103.61 g polyethylene glycol molecular weight 4000 g/mol, 3.53 g caprolactam (80% in water), and 7.25 g water are placed. To the mixture 2.60 g sulfuric acid (96%) is added within 10 minutes. Temperature rises up to 50° C. during sulfuric acid addition. The vessel is closed and heated to 148° C. bath temperature and stirred for 6 hours at this temperature. The reaction mixture is transferred to a 4-neck vessel with thermometer, nitrogen inlet, stirrer, and distillation head. Water is distilled off for 27 hours at 5 mbar and 130° C. bath temperature. 108.0 g of a brown solid is obtained. ¹H-NMR in MeOD indicates 50% conversion of hydroxyl groups into 6-aminohexane acid ester and 50% conversion of hydroxyl groups into sulfuric acid ester.

### Example 5: Polyethyleneglycol polypropyleneglycol Block Copolymer Pluronic PE 6400, Esterified with 1 Mole Caprolactam and Esterified with 1 Mole Sulfuric Acid

In a 4-neck vessel with thermometer, nitrogen inlet, dropping funnel, reflux condenser and stirrer 101.5 g polyethyleneglycol polypropyleneglycol block copolymer Pluronic PE 6400, 4.95 g caprolactam (80% in water), and 3.15 g water are placed. To the mixture 3.65 g sulfuric acid (96%) is added within 10 minutes. Temperature rises up to 50° C. during sulfuric acid addition. The reaction mixture is heated to 148° C. bath temperature and stirred for 6 hours at this temperature. The reflux condenser is replaced by a distillation head, and water is distilled off for 22 hours under vacuum up to 5 mbar. 107.0 g of a brown solid is obtained. ¹H-NMR in MeOD indicates 45% conversion of hydroxyl groups into 6-aminohexane acid ester and 44% conversion of hydroxyl groups into sulfuric acid ester.

### Example 6: Glycerol, Ethoxylated with 12 Mole Ethylene Oxide, Esterified with 1 Mole Caprolactam and Sulfatized with 1 Mole Sulfuric Acid

### 6a Glycerol, Ethoxylated with 12 Mole Ethylene Oxide

In a 2 l autoclave 110.5 g glycerol and 1.5 g potassium tert. butoxide are placed and the mixture is heated to 80° C. The vessel is purged three times with nitrogen and the mixture is heated to 140° C. 634.3 g ethylene oxide is added in portions within 11 hours. To complete the reaction, the mixture is allowed to post-react for additional 5 hours at 140° C. The reaction mixture is stripped with nitrogen and volatile compounds are removed in vacuo at 80° C. After filtration 745.0 g of a brown oil is obtained (hydroxy value: 85.0 mgKOH/g).

### 6b Glycerol, Ethoxylated with 12 Mole Ethylene Oxide, Esterified with 1 Mole Caprolactam and Sulfatized with 1 Mole Sulfuric Acid

In a 4-neck vessel with thermometer, nitrogen inlet, dropping funnel, reflux condenser and stirrer 62.2 g glycerol, ethoxylated with 12 mole ethylene oxide 14.1 g caprolactam (80% in water), and 9.0 g water are placed. To the mixture 10.3 g sulfuric acid (96%) is added within 10 minutes. Temperature rises to 60° C. during sulfuric acid addition. The reaction mixture is heated to 135° C. bath temperature and stirred for 6 hours at this temperature. The reflux condenser is removed and replaced by a distillation head. Water is distilled off for 5 hours under vacuum up to 5 mbar. 80.0 g of a brown solid is obtained. ¹H-NMR in MeOD indicates 32% conversion of hydroxyl groups into 6-aminohexane acid ester and 31% conversion of hydroxyl groups into sulfuric acid ester.

### Example 7: Pentaerythritol, Ethoxylated with 16 Mole Ethylene Oxide, Esterified with 1.3 Mole Caprolactam and Sulfatized with 1.3 Mole Sulfuric Acid

### 7a Pentaerythritol, Ethoxylated with 16 Mole Ethylene Oxide

In a 2 l autoclave 130.0 g pentaerythritol and 1.6 g potassium tert. butoxide and 300.0 ml xylene (mixture of isomers) are placed and the mixture is heated to 80° C. The vessel is purged three times with nitrogen and the mixture is heated to 140° C. 673.1 g ethylene oxide is added in portions within 6.5 hours. To complete the reaction, the mixture is allowed to post-react for additional 6 hours at 140° C. The reaction mixture is stripped with nitrogen and solvent xylene is removed in vacuo at 2 mbar at 120° C. After filtration 831.0 g of a yellow oil is obtained (hydroxy value: 271.0 mgKOH/g).

### 7b Pentaerythritol, Ethoxylated with 16 Mole Ethylene Oxide, Esterified with 1.3 Mole Caprolactam and Sulfatized with 1.3 Mole Sulfuric Acid

In a 4-neck vessel with thermometer, nitrogen inlet, dropping funnel, reflux condenser and stirrer 92.7 g pentaerythritol, ethoxylated with 16 mole ethylene oxide, 20.3 g caprolactam (80% in water), and 8.3 g water are placed. To the mixture 14.7 g sulfuric acid (96%) is added within 10 minutes. Temperature rises to 60° C. during sulfuric acid addition. The reaction mixture is heated to 135° C. bath temperature and stirred for 7 hours at this temperature. The reflux condenser is removed and replaced by a distillation head. Water is distilled off for 8 hours at 135° C. bath temperature. Vacuum (5 mbar) is applied, and the mixture is stirred for 5 under vacuum and 135° C. bath temperature. 80.0 g of a brown solid is obtained. ¹H-NMR in MeOD indicates 27% conversion of hydroxyl groups into 6-aminohexane acid ester and 32% conversion of hydroxyl groups into sulfuric acid ester.

### Example 8: Sorbitol, Propoxylated with 96 Mole Propylene Oxide and Ethoxylated with 144 Mole Ethylene Oxide, Esterified with 3 Mole Caprolactam and Sulfatized with 3 Mole Sulfuric Acid

### 8a Sorbitol, Propoxylated with 96 Mole Propylene Oxide and Ethoxylated with 144 Mole Ethylene Oxide

In a 3 l autoclave 140.0 g of a sorbitol propoxylate, propoxylated with 6.6 mole propylene oxide (Lupranol 3422, commercially available from BASF SE) and 5.0 g potassium butoxide are placed and the mixture is heated to 60° C. The vessel is purged three times with nitrogen and heated to 140° C. 1060.4 g propylene oxide is added in portions within 6 hours. To complete the reaction, the mixture is allowed to post-react for additional 6 hours at 140° C. 1295.3 g ethylene oxide is added within 6 hours at 140° C., followed by post-reaction time of 6 hours at 140° C. The reaction mixture is stripped with nitrogen and volatile compounds are removed in vacuo at 80° C. After filtration 2490.0 g of a waxy brown solid is obtained (hydroxy value: 33.6 mgKOH/g).

### 8b Sorbitol, Propoxylated with 96 Mole Propylene Oxide and Ethoxylated with 144 Mole Ethylene Oxide, Esterified with 3 Mole Caprolactam and Sulfatized with 3 Mole Sulfuric Acid

In a 4-neck vessel with thermometer, nitrogen inlet, dropping funnel, reflux condenser and stirrer 122.5 g sorbitol, propoxylated with 96 mole propylene oxide and ethoxylated with 144 mole ethylene oxide (8 a), 4.24 g caprolactam (80% in water), and 1.8 g water are placed. To the mixture 3.11 g sulfuric acid (96%) is added within 10 minutes under a constant stream of nitrogen. Temperature rises up to 55° C. during sulfuric acid addition. The reaction mixture is heated to 135° C. bath temperature and is stirred for 7 hours at 135° C. under reflux. The reflux condenser is removed and under a constant stream of nitrogen, water is distilled off for 3 hours. The reaction mixture is stirred at 130° C. for 9 hours under vacuum (<25 mbar). 127.0 g of a brown solid is obtained. ¹H-NMR in MeOD indicates 49% conversion of hydroxyl groups into 6-aminohexane acid ester and 47% conversion of hydroxyl groups into sulfuric acid ester.

### Example 9: Sorbitol, Propoxylated with 96 Mole Propylene Oxide and Ethoxylated with 144 Mole Ethylene Oxide, Esterified with 1 Mole Caprolactam and Sulfatized with 1 Mole Sulfuric Acid

In a 4-neck vessel with thermometer, nitrogen inlet, dropping funnel, reflux condenser and stirrer 121.13 g sorbitol, propoxylated with 96 mole propylene oxide and ethoxylated with 144 mole ethylene oxide (1 c), 1.41 g caprolactam (80% in water), and 0.09 g water are placed. To the mixture 1.03 g sulfuric acid (96%) is added within 10 minutes under a constant stream of nitrogen. Temperature rises up to 55° C. during sulfuric acid addition. The reaction mixture is heated to 135° C. bath temperature and is stirred for 29 hours at 135° C. under reflux. The reflux condenser is removed and under a constant stream of nitrogen, water is distilled off for 3 hours. The reaction mixture is stirred at 130° C. for 6 hours under vacuum (<25 mbar). 120.0 g of a brown solid is obtained. ¹H-NMR in MeOD indicates 15% conversion of hydroxyl groups into 6-aminohexane acid ester and 15% conversion of hydroxyl groups into sulfuric acid ester.

### Example 10: Sorbitol, Propoxylated with 6.6 Mole Propylene Oxide and Ethoxylated with 23.4 Mole Ethylene Oxide, Esterified with 3 Mole Caprolactam and Sulfatized with 3 Mole Sulfuric Acid

### 10a Sorbitol, Propoxylated with 6.6 Mole Propylene Oxide and Ethoxylated with 23.4 Mole Ethylene Oxide

In a 2 l autoclave 354.0 g of a sorbitol propoxylate, (Sorbitol propoxylated with 6.6 mole propylene oxide (Lupranol 3422, commercially available from BASF SE)) and 1.8 g potassium butoxide are placed and the mixture is heated to 60° C. The vessel is purged three times with nitrogen and heated to 140° C. 532.3 g ethylene oxide is added in portions within 6 hours. To complete the reaction, the mixture is allowed to post-react for additional 6 hours at 140° C. The reaction mixture is stripped with nitrogen and volatile compounds are removed in vacuo at 110° C. After filtration 874.0 g of a brown oil is obtained (hydroxy value: 199.8 mgKOH/g).

### 10b Sorbitol, Propoxylated with 6.6 Mole Propylene Oxide and Ethoxylated with 23.4 Mole Ethylene Oxide, Esterified with 3 Mole Caprolactam and Sulfatized with 3 Mole Sulfuric Acid

In a 4-neck vessel with thermometer, nitrogen inlet, dropping funnel, reflux condenser and stirrer 68.8 g sorbitol, propoxylated with 6.6 mole propylene oxide and ethoxylated with 23.4 mole ethylene oxide (10 a), 16.9 g caprolactam (80% in water), and 6.5 g water are placed. To the mixture 12.5 g sulfuric acid (96%) is added within 10 minutes under a constant stream of nitrogen. Temperature rises up to 55° C. during sulfuric acid addition. The reaction mixture is heated to 135° C. bath temperature and is stirred for 8 hours at 135° C. under reflux. The reflux condenser is removed and under a constant stream of nitrogen, water is distilled off for 3 hours. The reaction mixture is stirred at 130° C. for 8 hours under vacuum (<25 mbar). 90.0 g of a brown solid is obtained. ¹H-NMR in MeOD indicates 33% conversion of hydroxyl groups into 6-aminohexane acid ester and 41% conversion of hydroxyl groups into sulfuric acid ester.

### Example 11: Sorbitol, Propoxylated with 6.6 Mole Propylene Oxide and Ethoxylated with 113.4 Mole Ethylene Oxide, Esterified with 1 Mole Caprolactam and Sulfatized with 1 Mole Sulfuric Acid

### 11a Sorbitol, Propoxylated with 6.6 Mole Propylene Oxide and Ethoxylated with 113.4 Mole Ethylene Oxide

In a 2 l autoclave 300.0 g of a sorbitol alkoxylate (10 a) and 1.4 g potassium butoxide are placed and the mixture is heated to 60° C. The vessel is purged three times with nitrogen and heated to 140° C. 691.6 g ethylene oxide is added in portions within 6 hours. To complete the reaction, the mixture is allowed to post-react for additional 6 hours at 140° C. The reaction mixture is stripped with nitrogen and volatile compounds are removed in vacuo at 80° C. After filtration 990.0 g of a brown oil is obtained (hydroxy value: 64.7 mgKOH/g).

### 11b Sorbitol, Propoxylated with 6.6 Mole Propylene Oxide and Ethoxylated with 113.4 Mole Ethylene Oxide, Esterified with 1 Mole Caprolactam and Sulfatized with 1 Mole Sulfuric Acid

In a 4-neck vessel with thermometer, nitrogen inlet, dropping funnel, reflux condenser and stirrer 85.4 g sorbitol, propoxylated with 6.6 mole propylene oxide and ethoxylated with 113.4 mole ethylene oxide (11 b), 2.1 g caprolactam (80% in water), and 0.9 g water are placed. To the mixture 1.6 g sulfuric acid (96%) is added within 10 minutes under a constant stream of nitrogen. Temperature rises up to 55° C. during sulfuric acid addition. The reaction mixture is heated to 135° C. bath temperature and is stirred for 6 hours at 135° C. under reflux. The reflux condenser is removed and under a constant stream of nitrogen, water is distilled off for 3 hours. The reaction mixture is stirred at 130° C. for 8 hours under vacuum (<25 mbar). 85.0 g of a brown solid is obtained. ¹H-NMR in MeOD indicates 15% conversion of hydroxyl groups into 6-aminohexane acid ester and 15% conversion of hydroxyl groups into sulfuric acid ester.

### Example 12: Sorbitol, Propoxylated with 6.6 Mole Propylene Oxide and Ethoxylated with 113.4 Mole Ethylene Oxide, Esterified with 3 Mole Caprolactam and Sulfatized with 3 Mole Sulfuric Acid

In a 4-neck vessel with thermometer, nitrogen inlet, dropping funnel, reflux condenser and stirrer 85.4 g sorbitol, propoxylated with 6.6 mole propylene oxide and ethoxylated with 113.4 mole ethylene oxide (11 a), 6.3 g caprolactam (80% in water), and 0.9 g water are placed. To the mixture 4.7 g sulfuric acid (96%) is added within 10 minutes under a constant stream of nitrogen. Temperature rises up to 55° C. during sulfuric acid addition. The reaction mixture is heated to 135° C. bath temperature and is stirred for 8 hours at 135° C. under reflux. The reflux condenser is removed and under a constant stream of nitrogen, water is distilled off for 3 hours. The reaction mixture is stirred at 130° C. for 7 hours under vacuum (<25 mbar). 89.0 g of a brown solid is obtained. ¹H-NMR in MeOD indicates 40% conversion of hydroxyl groups into 6-aminohexane acid ester and 42% conversion of hydroxyl groups into sulfuric acid ester.

### Comparative Example 1: Sorbitol, Propoxylated with 96 Mole Propylene Oxide and Ethoxylated with 144 Mole Ethylene Oxide, Esterified with 2 Mole Caprolactam as Methane Sulfonic Acid Salt

In a 4-neck vessel with thermometer, nitrogen inlet, dropping funnel, reflux condenser and stirrer 121.1 g sorbitol, propoxylated with 96 mole propylene oxide and ethoxylated with 144 mole ethylene oxide (1 c), 2.8 g caprolactam (80% in water), and 1.2 g water are placed. To the mixture 2.05 g methane sulfonic acid (99%) is added within 10 minutes under a constant stream of nitrogen. Temperature rises up to 40° C. during sulfuric acid addition. The reaction mixture is heated to 135° C. bath temperature and is stirred for 25 hours at 135° C. under reflux. The reflux condenser is removed and under a constant stream of nitrogen, water is distilled off for 7 hours. 120.0 g of a brown solid is obtained. ¹H-NMR in MeOD indicates 30% conversion of hydroxyl groups into 6-aminohexane acid ester as methane sulfonic acid salt.

### Performance in Detergents

For the whiteness benefit test, the following laundry detergent composition is provided in table1:

TABLE 1
laundry detergent composition

| Ingredient | % |
|---|---|
| LAS | 6.9 |
| AE3S | 11.3 |
| Fatty Acid C1218 | 1 |
| 1,2 Propylene Glycol | 6 |
| Ethanol | 2 |
| Water | balance |

### Test Preparation

The following fabrics are provided for the whiteness benefit test:
Polyester 1: Polyester 854, available from Reichenbach Wirkstoffe (Germany)
Polyester 2: PW19, available from Empirical Manufacturing Company (Cincinnati, OH, USA).
Knitted Cotton 1: CW120, available from Empirical Manufacturing Company (Cincinnati, OH, USA).

“Washed and FE Treated” fabrics were prepared according to the following method: 400 g fabrics are washed in a WE Miniwasher (3.5 litre water) twice using the short program (45 minute wash cycle followed by three rinse cycles; total program is 90 minutes) at 60° C. with 18.6 g Ariel Compact powder detergent, twice using the short program, at 60° C. nil detergent, and then three times using the short program at 40° C. with 8.2 g Lenor Concentrate (a fabric enhancer) into each main wash. Fabrics are then dried in a tumble dryer on extra dry until dry.

“Washed” fabrics were prepared according to the following method: 400 g fabrics are washed in a WE Miniwasher (3.5 litre water) twice using the short program (45 minute wash cycle followed by three rinse cycles; total program is 90 minutes) at 60° C. with 18.6 g Ariel Compact powder detergent and twice using the short program, at 60° C. nil detergent. Fabrics are then dried in a tumble dryer on extra dry until dry.

### Test Method

Four fabric samples are prepared: Polyester 1, washed and FE treated; Polyester 2, washed and FE treated; Knitted Cotton 1, washed and FE treated; Knitted Cotton 2, washed.

Each sample is run in a 96 well plate simulated washing system that uses magnetized bearings to simulate the agitation of a typical full scale washing machine according to the following conditions: 375 ppm detergent concentration, 150 μL water per well, 25° C., water hardness of 1.0 mM (2:1 Ca+2: Mg+2 molar ratio), wash pH of 8, 3000 ppm Arizona test dust (supplied PTI, Powder Technology Inc).

Each polymer (example 1 and comparative example 1) listed in table 2 is added at 100 ppm of the wash solution. Each fabric is washed for 60 minutes and dried in the dark under ambient conditions. For each wash condition, there are two 96 well plates, and eight internal replicates per 96 well plate, for a total of 16 replicates per wash condition. When the samples are dry, L*, a*, b* and CIE WI are measured on each 96 well plate spot using a Spectrolino imaging system (Gretag Macbeth, Spectro Scan 3.273). For each treatment, the average CIE WI is determined. Delta CIE WI, as reported in table 2 below, is the difference of the average CIE WI of the sample vs. the average CIE WI of a control sample without the tested polymer.

TABLE 2
Whiteness benefits of example 1 and comparative example 1

| | Delta CIE WI | | | |
|---|---|---|---|---|
| | Polyester 1, | Polyester 2, | Knitted Cotton | Knitted |
| | washed and | washed and | 1, washed and | Cotton |
| | FE treated | FE treated | FE treated | 1, washed |
| Example 1 | 6.4 | 6.4 | 1.2 | 9.4 |
| Comparative | 2.6 | 3.8 | −3.7 | 6.1 |
| Example 1 | | | | |
| LSD (95) | 1.7 | 1.6 | 1.3 | 1.2 |

1.-17. (canceled)

18. A sulfatized esteramine obtained by a process comprising step a):
a) reacting at least one alcohol containing at least two hydroxy groups (compound (A)) with at least one lactam (compound (B)) and with sulfuric acid (compound (C)).

19. The sulfatized esteramine according to claim 18, wherein within step a)
i) at least a fraction of compound (A) is first mixed with at least a fraction of compound (B) followed by continuously adding at least a fraction of compound (C) over a specific period of time, and/or
ii) compound (C) is added for a specific period of time and the specific period of time for continuously adding compound (C) is in the range of less than one hour, and/or
iii) the reaction is carried out after all compounds (A) to (C) are admixed with each other at a temperature of 80 to 200° C. and/or water is removed from the reaction mixture.

20. The sulfatized esteramine according to claim 18, wherein compound (A) is at least one alcohol containing at least two hydroxy groups selected from diols, polyols, alkoxylated diols and alkoxylated polyols.

21. The sulfatized esteramine according to claim 18, wherein in case compound (A) comprises an alkoxylated alcohol containing at least two hydroxy groups, the alkoxylated fragment of the respective alcohol is based on at least one C₂-C₂₂ alkylene oxide.

22. The sulfatized esteramine according to claim 18, wherein the process comprises step b), which is carried out prior to step a):
b) at least one alcohol containing at least two hydroxy groups and having a molecular weight M_{W} of less than 500 g/mol is reacted with at least one alkylene oxide in order to obtain an alkoxylated alcohol as compound (A).

23. The sulfatized esteramine according to claim 22, wherein
i) ethylene oxide and/or propylene oxide is employed, and/or
ii) at least one alcohol containing at least two hydroxy groups and having a molecular weight M_{W} of less than 500 g/mol is reacted with at least 1 mol of propylene oxide and/or with at least 1 mol of ethylene oxide, and/or
iii) at least one alcohol containing at least two hydroxy groups and having a molecular weight M_{W} of less than 500 g/mol is reacted batchwise with ethylene oxide and/or propylene oxide in order to obtain at least one block based on ethylene oxide and/or propylene oxide on the respective alkoxylated alcohol, and/or
iv) at least one alcohol containing at least two hydroxy groups and having a molecular weight M_{W} of less than 500 g/mol is reacted in at least one batch with 1 to 120 mol of propylene oxide followed by at least one batch of 1 to 150 mol ethylene oxide.

24. The sulfatized esteramine according to claim 18, wherein
i) step a) is carried out in the presence of water, and/or
ii) optionally carrying out a step c) by removing water and/or by removing excess alcohol according to compound (A).

25. The sulfatized esteramine according to claim 18, wherein in step a) the molar ratio of compound (C) to compound (B) is at least 100 mol-%.

26. The sulfatized esteramine according to claim 18, wherein in step a) the molar ratio of compound (C) to compound (B) is at least 90 mol-%.

27. The sulfatized esteramine according to claim 18, wherein in step a) the molar ratio of compound (B) to the hydroxy groups of compound (A) is in the range of 10 mol-% to 50 mol-%.

28. The sulfatized esteramine according to claim 18, wherein in step a) the molar ratio of compound (C) to the hydroxy groups of compound (A) is in the range of 10 mol-% to 62,5 mol-%.

29. The sulfatized esteramine according to claim 18, wherein in step a) at least 10% of all hydroxy groups of compound (A) are reacted with compound (B) in order to form ester groups within the respective sulfatized esteramine and/or at least 10% of all hydroxy groups of compound (A) are sulfatized in order to form OSO₃ fragments within the respective sulfatized esteramine.

30. The sulfatized esteramine according to claim 29, wherein in step a)
20 to 50% of all hydroxy groups of compound (A) are reacted with compound (B) in order to form ester groups within the respective sulfatized esteramine,
20 to 50% of all hydroxy groups of compound (A) are sulfatized in order to form OSO₃ fragments within the respective sulfatized esteramine, and
0 to 30% of all hydroxy groups of compound (A) remain in unreacted form within the respective sulfatized esteramine.

31. The sulfatized esteramine according to claim 18, wherein within step a)
i) the reaction is carried out after all compounds (A) to (C) are admixed with each other at a temperature of 80 to 200° C. for a period of time of 1 to 30 hours, and/or
ii) the reaction is carried out in a closed vessel under pressure from 1.0 up to 10 bar.

32. The sulfatized esteramine according to claim 18, wherein compound (B) is at least one ε-lactam.

33. A process for producing the sulfatized esteramine according to claim 18, wherein the process comprises step a):
a) reacting at least one alcohol containing at least two hydroxy groups (compound (A)) with at least one lactam (compound (B)) and with sulfuric acid (compound (C)).

34. Sulfatized esteramines of Formula (I) and salts thereof, 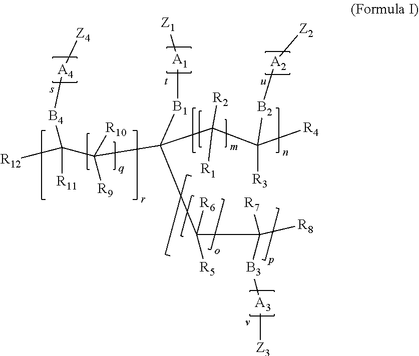
wherein independently from each other
n being an integer from 1 to 12,
m being an integer for each repetition unit n independently selected from 0 to 12;
p being an integer from 0 to 12,
o being an integer for each repetition unit p independently selected from 0 to 12;
r being an integer from 0 to 12,
q being an integer for each repetition unit r independently selected from 0 to 12;
s, t, u and v being an integer from 0 to 100;
A₁, A₂, A₃, and A₄ are independently from each other and independently for each repetition unit s, t, u, or v, selected from the list consisting of alkyleneoxy group, such A-units stem from the reaction of one alcohol with at least two hydroxy groups with C2-C22 alkylene oxides, e.g. in case of ethoxylated alcohols with at least two hydroxy groups A is “—O—CH2—CH2—”
wherein for s, t, u, and/or v equal to 1 the oxygen atom of the A₁, A₂, A₃, and A₄ group is bound to the B group and the following A₁, A₂, A₃, and A₄ groups are always bound via the oxygen atom to the previous A₁, A₂, A₃, and A₄ group.
B₁, B₂, B₃, and B₄ are independently from each other selected from the group consisting of a bond, linear C₁ to C₁₂ alkanediyl groups, and branched C₁ to C₁₂ alkanediyl groups;
such B-units are given by the molecular structure of one alcohol with at least two hydroxy groups, e.g. in case of example 2 (1,6-hexane diol, esterified with 1 mol caprolactam and esterified with 1 mole sulfuric acid) B1 and B2 are “—CH2—”, with p and r=0, n=1, m=2, t and u=0, R₁, R₂, R₃ R₄, R₈, R₉, and R₁₂=H, Z1 and Z2=OSO3H, OH or Formula (II) with w=3, and R₁₃, R₁₄ R₁₅, R₁₆, R₁₇, and R₁₈=H,
R₁, R₂, R₃ R₄, R₅, R₆, R₇, R₈, R₉, R₁₀, R₁₁ and R₁₂ being independently for each repetition unit selected from the group consisting of H, linear alkyl, branched alkyl, and cycloalkyl; such R-units are given by the molecular structure of one alcohol with at least two hydroxy groups,
Z₁, and/or Z₂, and/or Z₃, and/or Z₄, independently for each repetition unit n, p, and r, are selected from the group consisting of OH, and OSO3—, and —OSO3H and a compound according to Formula (II), wherein said compound according to Formula (II) connects to the compound according to Formula (I) via the bond labeled with *, such Z-units stem from the reaction of one alcohol with at least two hydroxy groups with at least one lactam and with sulfuric acid, e.g. in case of reaction with C4 lactam and sulfuric acid, Z₁, Z₂, Z₃, Z₄, are “—OC(O)—CH₂—CH₂—CH₂—NH₂ or SO₃H or OH
with the provisio that at least 10 mol % to 50 mol % of the substituents Z₁, and/or Z₂, and/or Z₃, and/or Z₄, are a compound according to Formula (II), and at least 10 mol % to 50 mol % of the substituents Z₁, and/or Z₂, and/or Z₃, and/or Z₄, are a group consisting OSO3—, or —OSO3H, and 0 mol % to 80 mol % of the substituents Z₁, and/or Z₂, and/or Z₃, and/or Z₄, are OH, 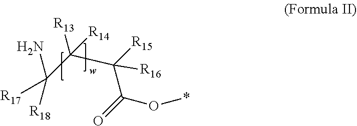
with independently from each other
w being an integer from 0 to 12;
R₁₃, R₁₄ R₁₅, R₁₆, R₁₇, and R₁₈ independently being selected from the group consisting of H, linear alkyl, branched alkyl, and cycloalkyl; such R-units stem from the lactam, e.g. in case of reaction with C4 lactam R₁₃, R₁₄ R₁₅, R₁₆, R₁₇, and R₁₈ are=H, w=1, and then Formula (II) is “—OC(O)—CH₂—CH₂—CH₂—NH₂”
